(19) United States
(12) Patent Application Publication
(10) Pub. No.: US 2011/0015602 A1
(43) Pub. Date: Jan. 20, 2011

(54) Hydrophilic Nonwovens with Low Retention Capacity Comprising Cross-Linked Hydrophilic Polymers

(76) Inventors: Mattias Schmidt, Idstein (DE); Stephen Allen Goldman, Montgomery, OH (US); Giovanni Carrara, Montesilvano (IT); Ekaterina Anatolyevna Ponomarenko, Bad Soden (DE)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/892,265

(22) Filed: Sep. 28, 2010

Related U.S. Application Data
(63) Continuation of application No. 11/089,272, filed on Mar. 24, 2005.

(51) Int. Cl. A61F 13/15; B32B 27/00; D04H 13/00; B29C 71/04

(52) U.S. Cl. 604/367; 442/118; 427/595

(57) ABSTRACT

A method for making hydrophilic synthetic nonwoven webs. At least a region of 1 cm by 1 cm comprised by the web may comprise cross-linked hydrophilic polymers. The web may have a retention capacity of less than 100 g of aqueous liquid per m² of the nonwoven fibrous web. An absorbent article comprising a hydrophilic synthetic nonwoven web.

### CROSS REFERENCE TO RELATED APPLICATIONS

This application is a divisional application of U.S. application Ser. No. 11/089,272, filed on Mar. 24, 2005 and claims the benefit of U.S. Provisional Application No. 60/557,171, filed Mar. 29, 2004, both of which are herein incorporated by reference in their entireties.

### FIELD OF THE INVENTION

The present invention relates to synthetic nonwoven webs comprising cross-linked hydrophilic polymers. The webs of the invention have retention capacities of less than 100 g of aqueous liquid per m² of the nonwoven fibrous web. Moreover, the invention relates to a method for making such nonwoven webs. Furthermore, the invention also relates to absorbent articles comprising the nonwoven webs comprising cross-linked hydrophilic polymers.

### BACKGROUND OF THE INVENTION

Nonwoven fabrics made of synthetic fibers are used for a wide variety of different applications, e.g., they are commonly applied in absorbent articles, for example, as topsheet material or as core wrap to enclose the storage layer of the absorbent core. Such nonwoven fabrics are usually hydrophobic. However, for many applications in hygiene products it is necessary to have hydrophilic nonwoven. Therefore, the nonwoven fabric has to be treated accordingly.

A common method for rendering nonwoven fabrics hydrophilic is coating the surface of the nonwoven with hydrophilic surfactants. As this coating does not lead to a tight, chemical bond between the nonwoven and the surfactant, the surfactant can be washed off during use when the absorbent article is wetted. The decrease in liquid strike through time is a desirable effect when the nonwoven is coated with surfactant. Liquid strike through refers to liquid passing through the nonwoven fabric with liquid strike through time referring to the time it takes for a certain amount of liquid to pass through the nonwoven. However, as the surfactant is washed off when coated nonwoven fabrics are exposed to liquid, the strike through time in the next gush(es) is increased again. This results in performance reduction during use for diapers comprising hydrophobic nonwoven fabrics treated with surfactants. Furthermore, at the same time as liquid strike through time decreases due to use of surfactants, the surface tension of the wash off (=liquid, which was in contact with the surfactant-treated nonwoven fabric) is reduced. This reduction is undesirable, because it can cause increased urine leakage in a diaper.

Another possibility to render a nonwoven fabric hydrophilic is by applying high energy treatment, such as corona treatment. Corona discharge is an electrical phenomenon, which occurs when air is exposed to a voltage potential high enough to cause ionization, thereby changing it from an electrical insulator to a conductor of electricity. However, corona treatment leads to low coating durability upon storage of material, i.e., hydrophilicity decreases over time.

Thus, there is a need for a hydrophilic coating of a nonwoven, which is durable upon storage, is not easily washed off when wetted and allows achieving fast liquid strike through in multiple exposures to liquid without significant surface tension reduction of wash-off.

Methods of chemically grafting hydrophilic monomers are known in the art. For example U.S. Pat. No. 5,830,604 entitled “Polymeric sheet and electrochemical device using the same” issued to Raymond et al.; U.S. Pat. No. 5,922,417 entitled “Polymeric sheet” issued to Raymond et al.; and WO 98/58108 entitled “Non-woven fabric treatment” all refer to a process to produce nonwovens for use as separator in electrochemical devices such as batteries.

These methods for chemically grafting require a washing step due to the relatively high amounts of unreacted monomers and soluble non-grafted polymers, which otherwise remain on the surface of the nonwoven fabric. If the unreacted monomers are not washed off properly, they may be washed off later on during use. In case the nonwoven fabrics are applied in absorbent articles, such monomers are highly undesirable, as they may come into contact with the wearer of the absorbent article. If the soluble polymers are not washed off, these may be highly swollen and/or washed off during use leading to a reduction in liquid strike through times. However, an additional washing step is relatively expensive, especially due to the high amounts of washing water, which has to be disposed, and the energy required for drying.

Additionally, it is well known in the art to produce nonwoven fabrics comprising superabsorbent polymers. For example, U.S. Pat. No. 6,417,425 entitled “Absorbent article and process for preparing an absorbent article” issued to Whitmore et al., discloses a process, which includes spraying onto a fibrous web a blend containing superabsorbent polymer particles, superabsorbent forming monomers, initiator and water. The web is then subjected to polymerization conditions.

Furthermore, U.S. Pat. No. 5,567,478 entitled “Process for producing a water-absorbing sheet material and the use thereof” issued to Houben et al. refers to a process for producing water-absorbing sheet-like materials which consist of a water-absorbent polymer and a prefabricated nonwoven fabric, wherein the prefabricated nonwoven fabric is impregnated with a solution comprising partially neutralized acrylic acid and at least one cross-linking agent and is squeezed to a certain coating amount, and the monomer solution thus applied is characterized in that the polymerization is carried out in the presence of radical initiators. The resulting product is said to have improved water absorption under load, and a higher retention.

The nonwoven webs produced by the processes disclosed in U.S. Pat. No. 6,417,425 and U.S. Pat. No. 5,567,478 are intended to have increase retention capacities. Therefore, the add-on levels of superabsorbent polymers onto the fibrous webs are relatively high. However, such nonwoven webs are not suitable as topsheet or acquisition layer material, as the superabsorbent polymers comprised by the nonwovens may easily block the pores of the webs when they are swollen upon absorption of liquid, thereby leading to reduced permeability for liquid strike through.

### SUMMARY OF THE INVENTION

A method for treating a synthetic nonwoven fibrous web to comprise cross-linked hydrophilic polymers may comprise providing a synthetic nonwoven fibrous web. The method may comprise providing an aqueous solution comprising hydrophilic monomers, cross-linker molecules and radical polymerization initiator molecules. The method may comprise contacting the web with the aqueous solution, such that the web is penetrated by the aqueous solution. The method may comprise exposing the web/plurality of fibers to UV radiation. The concentration of the monomers in the aqueous solution may be selected such that the add-on level of the hydrophilic polymers on the nonwoven web is less than 30% by weight of the web without said hydrophilic polymers.

### BRIEF DESCRIPTION OF THE DRAWINGS

While the specification concludes with claims pointing out and distinctly claiming the present invention, it is believed the same will be better understood by the following drawings taken in conjunction with the accompanying specification wherein like components are given the same reference number.

FIG. 1 is a top plan view of a disposable diaper, with the upper layers partially cut away.

FIG. 2 is a cross-sectional view along the transverse axis of the disposable diaper shown in FIG. 1

### DETAILED DESCRIPTION OF THE INVENTION

### Definitions

As used herein, the following terms have the following meanings:

“Absorbent article” refers to devices that absorb and contain liquid. In one embodiment, the term “absorbent article” refers to devices that are placed against or in proximity to the body of the wearer to absorb and contain the various exudates discharged from the body. Absorbent articles include but are not limited to diapers, adult incontinent briefs, training pants, diaper holders and liners, sanitary napkins and the like. Additionally, in another embodiment according to the present invention the term “absorbent articles” refers to wipes.

“Disposable” is used herein to describe articles that are generally not intended to be laundered or otherwise restored or reused i.e., they are intended to be discarded after a single use and, preferably, to be recycled, composted or otherwise disposed of in an environmentally compatible manner.

“Comprise,” “comprising,” and “comprises” is an open ended term that specifies the presence of what follows e.g., a component but does not preclude the presence of other features, elements, steps or components known in the art, or disclosed herein.

The term “hydrophilic” describes fibers or surfaces of fibers, which are wettable by aqueous fluids (e.g., aqueous body fluids) deposited on these fibers. Hydrophilicity and wettability are typically defined in terms of contact angle and the strike through time of the fluids, for example through a nonwoven fabric. This is discussed in detail in the American Chemical Society publication entitled “Contact angle, wettability and adhesion”, edited by Robert F. Gould (Copyright 1964). A fiber or surface of a fiber is said to be wetted by a fluid (i.e., hydrophilic) when either the contact angle between the fluid and the fiber, or its surface, is less than 90°, or when the fluid tends to spread spontaneously across the surface of the fiber, both conditions are normally co-existing. Conversely, a fiber or surface of the fiber is considered to be hydrophobic if the contact angle is greater than 90° and the fluid does not spread spontaneously across the surface of the fiber.

The terms “nonwoven fabric” and “nonwoven web” are used interchangeably.

The term “plurality of fibers” refers to plurality of individual fibers or filaments, which have not yet been transformed into a nonwoven web. However, the fibers may be entangled with each other. In a preferred embodiment of the invention, the plurality of fibers consists of continuous filaments.

### Nonwoven Fabrics

A nonwoven fabric is a manufactured web of directionally or randomly orientated fibers, bonded by friction, and/or cohesion and/or adhesion, excluding paper and products which are woven, knitted, tufted, stitch-bonded incorporating binding yams or filaments, or felted by wet-milling, whether or not additionally needled.

The fibres may be of natural or man-made origin. They may be staple or continuous filaments or be formed in situ.

Nonwoven fabrics can be formed by many processes such as meltblowing, spunbonding, carding. The basis weight of nonwoven fabrics is usually expressed in grams per square meter (g/m²).

Commercially available fibers have diameters ranging from less than about 0.001 mm to more than about 0.2 mm and they come in several different forms: short fibers (known as staple, or chopped), continuous single fibers (filaments or monofilaments), untwisted bundles of continuous filaments (tow), and twisted bundles of continuous filaments (yam). Fibers are classified according to their origin, chemical structure, or both. They can e.g., be made into fabrics (also called nonwovens, nonwoven webs or nonwoven fabrics).

The nonwoven fabrics may comprise fibers made by nature (natural fibers), made by man (synthetic fibers), or combinations thereof. Example natural fibers include but are not limited to: animal fibers such as wool, silk, fur, and hair; vegetable fibers such as cellulose, cotton, flax, linen, and hemp; and certain naturally occurring mineral fibers.

For use in the present invention, the nonwoven fabrics are synthetic. Synthetic fibers are man-made fibers, comprising fibers derived from natural sources and mineral sources. Example synthetic fibers, which are derived from natural sources include but are not limited to viscose, polysaccharides (such as starch, rayon and lyocell). Example fibers from mineral sources include but are not limited to polyolefin fibers such as polypropylene, polyethylene fibers and polyester. Fibers from mineral sources are derived from petroleum, and silicate fibers such as glass and asbestos.

Nonwoven webs can be formed by direct extrusion processes during which the fibers and webs are formed at about the same point in time, or by preformed fibers which can be laid into webs at a distinctly subsequent point in time. Example direct extrusion processes include but are not limited to: spunbonding, meltblowing, solvent spinning, electrospinning, and combinations thereof. Nonwoven webs often comprise several layers, which may e.g., be made of different extrusion processes.

As used herein, the term “spunbonded fibers” refers to small diameter fibers, which are formed by extruding molten thermoplastic material as filaments from a plurality of fine, usually circular capillaries of a spinneret. Spunbond fibers are quenched and generally not tacky when they are deposited onto a collecting surface. Spunbond fibers are generally continuous.

As used herein, the term “meltblown fibers” means fibers formed by extruding a molten thermoplastic material through a plurality of fine, usually circular, die capillaries as molten threads or filaments into converging high velocity gas (e.g., air) streams, which attenuate the filaments of molten thermoplastic material to reduce their diameter. Thereafter, the meltblown fibers are carried by the high velocity gas stream and are deposited on a collecting surface to form a web of randomly disbursed meltblown fibers.

Example “laying” processes include wet-laying and dry-laying. Example dry-laying processes include but are not limited to air-laying, carding, and combinations thereof typically forming layers. Combinations of the above processes yield nonwovens commonly called hybrids or composites.

The fibers in a nonwoven web are typically joined to one or more adjacent fibers at some of the overlapping junctions. This includes joining fibers within each layer and joining fibers between layers when there is more than one layer. Fibers can be joined by mechanical entanglement, by chemical bonds or by combinations thereof.

In a preferred embodiment of the present invention, the nonwoven fabric is made of polypropylene (PP) and/or polyethylene (PE) and/or polyester (PET). In another embodiment the nonwoven fabric is made of bicomponent fibers consisting of PP and PET or PE and PP.

For use as core wrap material in absorbent articles the nonwoven fabric is preferably made by a combination of spunbond and meltblown process (SMMS) and the basis weights are preferably from 7 g/m² to 30 g/m², more preferably from 8 g/m² to 20 g/m², and even more preferably from 8 g/m² to 15 g/m².

For use as topsheet material in absorbent articles, the nonwoven fabric preferably comprises spunbond fibers. The basis weight of the topsheet is preferably between 10 g/m² and 30 g/m², more preferably from 15 g/m² to 20 g/m². In another embodiment, the topsheet comprises a carded nonwoven fabric with preferred basis weights from 10 g/m² to 25 g/m², more preferably from 15 g/m² to 20 g/m².

For application as acquisition material in the absorbent articles, the nonwoven is preferably made by a carding process and the basis weights are preferably from 20 g/m² to 200 g/m², more preferably from 40 g/m² to 100 g/m² and even more preferably from 50 g/m² to 70 g/m². The material is further bonded, e.g., by resin-, or air-through thermal bonding processes.

### Process for Making Permanently Hydrophilic Nonwoven Fabrics

The process of the present invention refers to the treatment of a synthetic nonwoven webs or to the treatment of a plurality of synthetic fibers. The process is very economic, because it comprises relatively inexpensive chemicals. Furthermore, the process is very fast. It can be run at line speeds of at least 200 m/min, more preferably at least 300 m/min and even more preferably at least 400 m/min.

The process for treating nonwoven webs/plurality of fibers according to the present invention comprises the following steps:

### Step a)

Providing a synthetic nonwoven web or providing a plurality of synthetic fibers. The nonwoven web or the fibers may be made of resins like polyamide, polypropylene, polyethylenes, polyester or the like. The fibers comprised by the nonwoven web typically have diameters ranging from less than about 0.001 mm to more than about 0.2 mm.

Preferably, the basis weight of the nonwoven webs suitable for the present invention is from 5 g/m² to 200 g/m², more preferably from 7 g/m² to 150 g/m² and still more preferably from 7 g/m² to 100 g/m².

An especially preferred web for the present invention is a web with spunbond-meltblown-meltblown-spunbond layers (SMMS) consisting of polypropylene. Hence, the nonwoven is a multilayer web having two outer layers formed from spunbonding and two inner layer formed from meltblowing. This SMMS nonwoven web preferably has a basis weight from 8 g/m² to 15 g/m².

Another especially preferred web for the present invention is a spunbonded web consisting of polypropylene (PP) and having a basis weight from 15 g/m² to 20 g/m².

A still further web, that is especially preferred for the present invention, is a carded web consisting of polyester (PET) and having a basis weight from 40 g/m² to 80 g/m².

If a plurality of fibers is used for the method of the invention, the plurality of fibers can be formed into a nonwoven fabric in a further method step at any point of the method of the invention, for example before contacting the plurality of fibers with the aqueous solution or after exposing the plurality of fibers to UV radiation. Moreover, the additional method step of forming the individual fibers or filaments into a nonwoven fabric may comprise at least a first plurality of fibers and a second plurality of fibers, wherein the first plurality of fibers is different from said second plurality of fibers. This difference might for example be due to different hydrophilic monomers in the aqueous solution. In one embodiment of the invention, only the first plurality of fibers has been subjected to the method of the present invention (treated fibers). In this embodiment the nonwoven fabric formed from the different pluralities of fibers comprises treated and untreated fibers.

### Step b)

Providing an aqueous solution comprising hydrophilic monomers, cross-linking molecules and radical polymerization initiators.

The aqueous solution comprises monomers capable of polymerization via a free-radical polymerization reaction. The monomer molecules comprised by the aqueous solution preferably contain at least one unsaturated double bond. Preferably the monomers comprise a group, such as an amine or carboxylic acid group, which can react with an acid or base to form a salt.

Suitable monomers and co-monomers can be acidic, neutral, basic, or zwitterionic. Suitable strong-acid monomers include those selected from the group of olefinically unsaturated aliphatic or aromatic sulfonic acids such as 3-sulfopropyl(meth)acrylate, 2-sulfoethyl(meth)acrylate, vinylsulfonic acid, styrene sulfonic acid, allyl sulfonic acid, vinyl toluene sulfonic acid, methacrylic sulfonic acid and the like. Particularly preferred strong-acid monomers are 2-acylamido-2-methylpropanesulfonic acid, 3-sulfopropyl(meth)acrylate, 2-sulfoethyl(meth)acrylate. Suitable weak-acid monomers include those selected from the group of olefinically unsaturated carboxylic acids and carboxylic acid anhydrides such as acrylic acid, methacrylic acid, maleic acid, itaconic acid, crotonic acid, ethacrylic acid, citroconic acid, fumaric acid, β-sterylacrylic acid and the like. Particularly preferred weak-acid monomers are acrylic acid and methacrylic acid.

Further suitable monomers for use in the present invention, such as cation-containing monomers, acid-containing monomers and non-acid monomers, are disclosed in U.S. Pat. No. 6,380,456 (columns 11 and 12).

Preferably, the monomers are comprised by the aqueous solution in a concentration between 10% and 70% by weight of solution, more preferably between 15% and 50%, still more preferably between 20% and 40% and most preferred between 25% and 35% of monomers. Relatively high monomers concentrations contribute to the overall efficiency of the polymerization process, which takes place in the method step of UV radiation. Thereby, the amount of un-reacted monomers remaining on the nonwoven web after UV radiation can be reduced.

The aqueous solution further comprises a radical polymerization initiator. The initiator is capable of forming reactive radicals upon activation with light (photo-initiator). Since suitable photo-initiators generally are best activated by absorption of UV light, the initiators should preferably be able to absorb light in the UV-spectrum. The initiators should further be sufficiently soluble in the aqueous solution comprising the monomers.

Suitable photo-initiators for use in the present invention include type α-hydroxy-ketones and benzilidimethyl-ketals. Further, suitable photo-initiators include dimethoxybenzylphenone (available under the trade name of Irgacure 651 from Ciba Specialty Chemicals Inc., Switzerland). 2-hydroxy-2-methyl-propiophenone (available under the trade name of Darocur 1173C from Ciba Specialty Chemicals Inc., Switzerland), 1-hydroxycyclohexylphenylketone (available under the trade name Irgacure 184 from Ciba Speciality Chemicals Inc., Switzerland), and diethoxyacetophenone, and 2-hydroxy-4′-(2-hydroxyethoxy)-2-methyl-propiophenone (available under the trade name of Irgacure 2959 from Ciba Speciality Chemicals Inc., Switzerland). Darocur 1173C, Irgacure 2959 and Irgacure 184 are preferred photo-initiators. Irgacure 2959, Darocur 1173C and Irgacure 184 are particularly preferred. Combinations of photo-initiators can also be used.

Further examples of suitable radical polymerization initiators are benzophenone and its derivates, acetophenone, benzoyl peroxide or azobisisobutyronitrile (AIBN).

Either one specific initiator may be applied or, alternatively, combinations of two or more different initiators. If combinations of different initiators are used, it is preferred that they have their maximum UV absorption at different ranges within the UV spectrum. Moreover, the initiator has to be chosen such that it absorbs the UV light used for the UV radiation method step. In addition to the photo-initiator(s), the aqueous solution can also comprise one or more additional free-radical initiator(s) such as thermal initiators and redox free-radical initiators which do not require absorption of light for the formation of free radicals. Suitable alternative initiators are disclosed for example in U.S. Re. 32,649.

As already pointed out, the solubility of the initiator in the aqueous solution has to be taken into consideration. The solubility will e.g., depend on the selected monomers. In case benzophenone is used as initiator and acrylic acids are used as monomers, the neutralization degree of the acrylic acid has to be chosen accordingly: Benzophenone is less soluble in aqueous solutions of sodium acrylate (neutralized acrylic acid) versus acrylic acid. Thus, at an acrylic acid concentration of about 30% and a neutralization degree of about 70%, the solubility of benzophenone is limited to a concentration of about up to 0.1% by weight of the aqueous solution compared to a solubility of benzophenone of about 0.5% by weight at a neutralization degree of 0%. As an alternative to benzophenone, more water-soluble hydrogen-abstraction initiators (e.g., acetophenone) can be applied.

As a further example, Darocur 1173C has good solubility in aqueous solutions of acrylic acid at a neutralization degree of 0% neutralization, but has only limited solubility at a neutralization degree of 70%. In order to increase the solubility of the initiators in these solutions, Irgacure 2959 can be used instead of Darocur 1173C. Although slow to dissolve, concentrations of at least about 2.0% by weight of the aqueous solution can be obtained in concentrated solutions of acrylic acid neutralized up to 70%.

Generally, the concentration of the initiator molecules in the aqueous solution is preferably from 0.01% to 5% by weight of the aqueous solution, more preferred from 0.1% to 3% by weight and even more preferred from 0.5% to 2.5% by weight. However, the preferred concentration will depend on the selected initiator/initiators, the applied monomers and the degree of neutralization of the monomers (for those embodiments, where the monomers can be neutralized). Relatively high concentrations of initiators contribute to the overall efficiency of the polymerization process, which takes place in the method step of UV radiation. Thereby, the amount of un-reacted monomers remaining on the nonwoven web following a relatively low dose of UV radiation can be reduced.

The cross-linking molecules suitable for the present invention are preferably polyfunctional (e.g., di-, tri-, tetra-functional). The cross-linker preferably is a polyfunctional monomer having at least two reactive sites capable of co-polymerizing with the selected monomers and has to be sufficiently soluble in the aqueous solution comprising the monomers.

Suitable polyfunctional monomer cross-linkers include polyethyleneoxide di(meth)acrylates with varying PEG molecular weights, IRR280 (a PEG diacrylate available from UCB Chemical, Belgium), trimethylolpropane ethyoxyiate tri(meth)acrylate with varying ethyleneoxide molecular weights, IRR210 (an alkoxylated triacrylate; available from UCB Chemicals, Belgium), trimethyolpropane tri(meth)acrylate, divnylbenzene, pentaerythritol triacrylate, pentaeythritol triallyl ether, triallylamine, N,N-methylene-bis-acrylamide and other polyfunctional monomer cross-linkers known to the art. Preferred monomer cross-linkers include the polyfunctional diacrylates and triacrylates.

Although less preferred, it is also possible to use all or in part polyfunctional crosslinkers containing only one polymerizable monomer group, wherein the crosslink is formed by reaction of one or more of the additional functional groups comprised by the cross-linker with monomers incorporated into the polymer chain, or even no polymerizable monomer group wherein the polyfunctional cross-linker reacts with at least two functional groups incorporated by polymerization into the polymer chain. It is also possible to use all or in part cross-linkers that form cross-links without the formation of covalent bonds. These cross-linkers can interact with functional groups on the polymer chain via non-covalent bonding interactions such as electrostatic, complexation, hydrogen bonding and dispersion force interactions. Suitable crosslinkers of these types are described in U.S. Re. 32,649.

The preferred concentration of the cross-linking molecules in the aqueous solution is from 0.01% to 10% by weight of the aqueous solution, more preferably from 0.1% to 5% by weight. A relatively high concentration of cross-linking molecules will result in a reduced concentration of soluble polymer. It will also result in a rather stiff hydrophilic polymer network after cross-linking, which is preferred in the present invention, because it reduces the ability of the hydrophilic polymer network to swell upon absorption of liquid. Thereby, it is ensured that the retention capacity of the nonwoven web of the invention is low and the pores of the nonwoven are not considerably blocked upon absorption of liquid.

Optionally the aqueous solution further comprises a surfactant and/or an organic solvent to improve wetting the nonwoven web by the aqueous solution. Examples for suitable organic solvents are various alcohols with alkyl chains of different lengths and different degrees of branching.

In general, surfactants can be anionic, cationic and non-ionic. Examples of nonionic, anionic, cationic, ampholytic, zwitterionic and semi-polar nonionic surfactants are disclosed in U.S. Pat. No. 5,707,950 and U.S. Pat. No. 5,576,282. According to the present invention, the use of non-ionic surfactants, such as long chain (C₁₂ to C₂₂) ethoxylated alcohols, is preferred. Cationic surfactants are less preferred if acrylic acids are used as monomers, because these surfactants may react with the acrylic acid monomer and precipitate it from the aqueous solution. A suitable surfactant for use in the present invention is Neodol 91-6 (available from Shell International B.V., The Netherlands). Neodol 91-6 has 9-11 methylene groups in the chain and 6 CH₂CH₂—O groups in the headgroup.

Preferably, the surfactants applied in the present invention act as co-monomers and thus get co-polymerized into the hydrophilic polymer comprised by the nonwoven web upon UV radiation. Thereby, it is ensured, that the surfactants will not be washed off during use of the nonwoven web upon contact with liquid, e.g., when the nonwoven web is used in absorbent articles.

Any polymerizable surfactant that co-polymerizes with the non-surfactant monomers can be used. Preferred surfactant monomers have a reactivity ratio with one or more of the available non-surfactant monomers which promotes a high degree of incorporation into the polymer chain. Thus, for the preferred (meth)acrylic acid monomers, it is preferred to have surfactant monomers containing co-polymerizable (meth)acrylic acid, ester, or amide functional groups. Particularly preferred polymerizable surfactants are acrylic acid and methacrylic acid esters of alkylpolyoxyethylene surfactants. Suitable methacrylic acid ester surfactants are available under the trade names of LEM 23 and BEM 23 by BIMAX Chemicals, USA.

If acrylic acids are used as monomers, preferred co-monomer surfactants comprise acrylic groups. It is believed that these acrylic groups enhance the co-polymerization with the acrylic acid.

Preferred concentrations of surfactants are from 0.01% to 30% by weight of the aqueous solution, more preferably from 0.25% to 10%, still more preferred from 0.25 to 5% by weight and most preferred from 0.5% to 2% by weight.

### Step c)

Contacting the nonwoven web or the plurality of fibers with the aqueous solution comprising hydrophilic monomers, cross-linking molecules and radical polymerization initiator. The monomers are capable to undergo a radical polymerization process.

The step of contacting the nonwoven web/plurality of fibers with the aqueous solution is preferably carried out under inert gas atmosphere, e.g., nitrogen, to reduce access of oxygen to the reaction medium. Preferably, the residual oxygen concentration in the inert gas atmosphere is less than 100 ppm, more preferably less than 60 ppm.

It is critical for the invention, that the aqueous solution penetrates the nonwoven web/plurality of fibers to ensure that the components comprised by the aqueous solution are in close contact with the fibers. This can be done, e.g., by applying the aqueous solution using increased pressure (such as in slot-coating) or by applying a vacuum on one side of the web/plurality of fibers (preferably below the web/plurality of fibers) and applying the aqueous solution from the other side of the web/plurality of fibers (preferably from above the web/plurality of fibers), whereby the aqueous solution is soaked into the web/plurality of fibers.

However, it is further critical for the invention that the add-on level of the components comprised by the aqueous solution, especially the add-on level of the monomers, is relatively low.

According to the method of the present invention, the concentration of the monomers comprised by the aqueous solution has to be selected such, that the add-on level of hydrophilic cross-linked polymers, which are formed in the subsequent method step of UV radiation on the nonwoven web/plurality of fibers is less than 30% (by weight of the nonwoven web/plurality of fibers without hydrophilic cross-linked polymers). Preferably, the concentration of the monomers in the aqueous solution should be selected such, that the add-on level of hydrophilic cross-linked polymers on the nonwoven web/plurality of fibers is less than 25% by weight, still more preferred less than 20% by weight, even more preferably less than 15% by weight and most preferred less than 10% by weight. However, the add-on level of the hydrophilic cross-linked polymers should be at least 1% by weight, more preferably at least 2% by weight.

The add-on level of the hydrophilic cross-linked polymers will depend on the add-on level of monomers after contacting the nonwoven web/plurality of fibers with the aqueous solution (but before the step of UV radiation): The add-on level of monomers will be at least as much as the add-on level of the cross-linked hydrophilic polymer. However, the add-on level of monomer directly after the web/plurality of fibers has been contacted with the aqueous solution may be higher than the add-on level of hydrophilic cross-linked polymer, as a part of the monomers may get lost prior to UV radiation, for example by evaporation. However, during UV radiation the monomers present on the web/plurality of fibers will almost completely be incorporated into the hydrophilic cross-linked polymers, as the polymerization process is very efficient, with low amounts of unreacted monomers. Therefore, the difference between add-on level of monomers and add-on level of hydrophilic cross-linked polymers will be small and mainly due to monomer losses because of evaporation.

Moreover, it is desirable to achieve a homogenous application of the aqueous solution on the nonwoven web/plurality of fibers.

Suitable methods of contacting the nonwoven web/plurality of fibers with the aqueous solution are, e.g., kiss-roll coating or spraying. Both methods are well known in the art.

However, the preferred way to apply the aqueous solution onto the nonwoven web/plurality of fibers according to the present invention is by slot coating in contact application, which is well known in the art. Slot coating ensures that the aqueous solution thoroughly penetrates the nonwoven web/plurality of fibers.

The aqueous solution can also be sprayed on the nonwoven web/plurality of fibers. Like the kiss-roll coating, spraying enables low and easily controllable add-on level of aqueous solution, which is preferred in the present invention.

According to the present invention, it is not preferred, that the nonwoven web/plurality of fibers is contacted with the aqueous solution by directly putting the nonwoven web/plurality of fibers into a bath comprising the aqueous solution, because then it is very difficult to control the add-on level of the components of the aqueous solution onto the nonwoven web/plurality of fibers.

A preferred solvent for use in the aqueous solution according to the present invention does not interfere significantly with the absorption of UV light by the photo-initiator used to initiate the polymerization reaction. For a specific photo-initiator in the solvent, this can be achieved if the solvent does not absorb a significant amount of UV light relative to the light absorbed by the photo-initiator for the frequencies outputted by the UV light source and utilized by the photo-initiator to initiate free-radical polymerization. Preferably the solvent is completely transparent to UV radiation. Also, preferably the solvent does not undergo chemical reaction (e.g., hydrogen abstraction) when directly exposed to UV irradiation or in the presence of the “excited-state” photo-initiator or any of the resultant free radicals. In addition, the solvent preferably does not adversely affect the properties of the nonwoven web/plurality of fibers. An example for a suitable solvent is water.

To ease the wettability of the nonwoven web/plurality of fibers and thus, to support the penetration of the aqueous solution into the nonwoven web/plurality of fibers according to the method of the present invention, it is possible to subject the nonwoven web/plurality of fibers to a corona treatment prior to contacting it with the aqueous solution.

Corona discharge is an electrical phenomenon, which occurs when air is exposed to a voltage potential high enough to cause ionization, thereby changing it from an electrical insulator to a conductor of electricity. By subjecting the nonwoven web/plurality of fibers to a corona treatment, the surface energy of the nonwoven web/plurality of fibers is increased, which makes facilitates the wettability.

Suitable equipment for corona treatment of the nonwoven web/plurality of fibers is a Laboratory Corona Treater (Model# BD-20AC, manufactured by Electro-Technic Products Inc., USA). Commercial scale equipment is supplied e.g., by Corotec company. Corona treatment and suitable equipment is well known in the art, see e.g., U.S. Pat. No. 5,332,897. A corona energy dose between 0.2 W/(ft×ft×min) and 0.5 W/(ft×ft×min) is suitable to increase the surface energy of a polypropylene nonwoven web/plurality of fibers from about 30 mN/m to between 40 mN/m and 45 mN/m.

The surface tension of water, as an example for an aqueous solution, is about 72 mN/m. Consequently, the web/plurality of fibers after corona treatment is easier to wet with water.

Moreover, corona treatment also contributes to the formation of radicals within the nonwoven web/plurality of fibers. Without being bound by theory, it is believed that thereby the number of hydrophilic polymers, which become chemically grafted onto the nonwoven web/plurality of fibers, can be increased. This contributes to a durable attachment of the hydrophilic polymers on the web/plurality of fibers and to reduced danger of wash off upon contact with liquid during use of the web/plurality of fibers, e.g., when used in an absorbent article.

Alternatively or in addition to corona treatment, the aqueous solution can comprise surfactants to decrease the surface tension of the solution.

Alternatively or in addition to surfactants being comprised by the aqueous solution, the nonwoven web/plurality of fibers, which is provided for the method of the invention can be treated with surfactants prior to contacting it with the aqueous solution.

### Step d)

Exposure of the nonwoven web/plurality of fibers to UV radiation after contacting the nonwoven web with the aqueous solution.

In a preferred embodiment of the present invention, a standard medium mercury lamp emitting UV with a maximum transmission between 100 nm and 400 nm, depending on the photo-initiator(s), which has/have been selected. Suitable lamps are for example available from IST Metz GmbH, Neurtingen, Germany (e.g., the lamp type M350K2H). Preferred lamps are characterized by an energy output from 160 W/cm to 200 W/cm of length of the lamp.

The energy level required to perform the reaction depends e.g., on the particular monomer chemistry, the required add-on level, the thickness of the nonwoven web/plurality of fibers, the line speed and the distance between nonwoven web/plurality of fibers and energy source, the concentration of photo-initiator, and the desired degree of reduction of residual monomer.

In a preferred embodiment the nonwoven web/plurality of fibers is positioned at the smallest possible distance from the UV radiation source without melting, burning or otherwise damaging the fibers.

According to the present invention, it is preferred that the nonwoven web is irradiated with UV light on both of the major surfaces of the web. Hence, the UV lamps are preferably positioned opposite both surfaces of the web. Thereby, the efficiency of the UV radiation can be increased.

The nonwoven web/plurality of fibers is preferably exposed to a total UV dose of at least 300 mJ/cm², more preferably at least 450 mJ/cm², even more preferably at least 600 mJ/cm², still more preferably at least 650 mJ/cm² and most preferably at least 700 mJ/cm².

The step of exposing the nonwoven web/plurality of fibers to UV radiation is preferably carried out under inert gas atmosphere, e.g., nitrogen, to reduce access of oxygen to the reaction medium. Preferably, the residual oxygen concentration in the inert gas atmosphere is less than 100 ppm, more preferably less than 60 ppm.

Absorption of UV light by the photo-initiator results in the formation of free radicals (e.g., by bond cleavage) capable of initiating free radical polymerization reaction(s) of the monomers present in solution. The resultant cross-linked hydrophilic polymer(s) are not significantly washed-off when the nonwoven web/plurality of fibers of the invention is contacted with liquid during use (e.g., contacted with urine in embodiments, wherein the nonwoven web/plurality of fibers is used in absorbent articles). Without being bound by theory, it is believed that the retention of the cross-linked hydrophilic polymer by the nonwoven web/plurality of fibers results from a combination of (i) the insolubility resulting from cross-linking and (ii) the entanglement of the cross-linked hydrophilic polymer with the web/plurality of fibers. It is believed that, as the monomers and the initiator have been brought into close contact with the fibers comprised by the nonwoven web/plurality of fibers, the cross-linked polymers will form around the fibers, “embracing” the fibers, so they are “locked” onto the fibers. Although not essential, preferably, at least a part of the polymers will also be chemically grafted to at least a part of the fibers comprised by the nonwoven web/plurality of fibers.

As the add-on level of the components (e.g., initiator molecules, cross-linking molecules), especially the add-on level of the monomers, is relatively low, the add-on level of the cross-linked hydrophilic polymer will also be relatively low, preferably less than 30% by weight (of the nonwoven web/plurality of fibers without hydrophilic cross-linked polymers), more preferably less than 25% by weight and still more preferred less than 20% by weight, even more preferably less than 15% by weight and most preferred less than 10% by weight. However, the add-on level of the cross-linked hydrophilic polymers should be at least 1% by weight, more preferably at least 2% by weight. Thereby, no superabsorbent polymers having a high retention capacity are formed on the nonwoven web/plurality of fibers. Consequently, the retention capacity of the nonwoven web comprising fibers treated according to the method of the present invention will be relatively low. This ensures that the polymer will not to swell considerably upon absorption of water, thereby possibly blocking the pores of the nonwoven web, which would decrease the liquid permeability of the nonwoven web. Especially for applications of the nonwoven web, such as topsheet, core wrap or acquisition layer in absorbent articles, liquid permeability of the nonwoven web is required.

As a result, a highly hydrophilic nonwoven web/plurality of fibers has been obtained by the process. Furthermore, the nonwoven web/plurality of fibers is durably hydrophilic, because the hydrophilic polymers are cross-linked and/or entangled within the web and/or chemically grafted to fibers of the web such, that they are not washed off upon contact with liquid.

Optionally, the method of the invention may comprise a drying step after UV radiation to eliminate any aqueous solution (especially solvent) possibly remaining on the nonwoven web/plurality of fibers.

The absorbent capacity as well as the retention capacity of the nonwoven web according to the present invention depends on the (absorbent/retention) capacities of the nonwoven web (without the hydrophilic polymers) on the one side and on the (absorbent/retention) capacities of the hydrophilic polymers comprised by the treated web on the other side.

Nonwoven webs typically have a retention capacity, which is considerably lower than their absorption capacity. This means that, while nonwoven webs may absorb a relatively high amount of liquid—depending, e.g., on the caliper and the basis weight of the web—, they are typically not able to retain the absorbed liquid, e.g., under pressure.

Polymers, such as superabsorbent polymers, commonly have both, a high absorption capacity and a high retention capacity. The liquid is “locked away” in the polymers even under pressure. Therefore, superabsorbent polymers are typically applied in the storage components of absorbent articles.

However, for the present invention, the primary task of the hydrophilic polymers comprised by the nonwoven web is not, to store liquid but to render the nonwoven web hydrophilic. The nonwoven web comprising the hydrophilic polymers is more readily wettable than an untreated,—commonly hydrophobic—synthetic nonwoven web. Therefore, the nonwoven web of the present invention can acquire and transport liquids more effectively than an untreated web. Such characteristics are especially desirable for use of the web as topsheet, acquisition layer or core wrap in absorbent articles, as quick absorption reduces the risk of leakage.

Due to the relatively small add-on levels and the relatively high degree of cross-linking of the hydrophilic polymers on the nonwoven web, the absorption capacities and retention capacities of these hydrophilic polymers will also be relatively low. Furthermore, as set out above, the retention limit of the nonwoven web as such (without the hydrophilic polymers) is relatively low. Hence, the overall retention capacity of the web according to the invention is relatively low. This is in contrast to superabsorbent polymers formed on nonwoven webs, such as are known in the art, which have a high retention capacity due to the superabsorbent polymers.

The retention capacity of at least one region having the dimensions of 1 cm by 1 cm of the nonwoven web of the invention comprising hydrophilic polymers is less than 100 g/m² (grams per square meter of nonwoven web comprising hydrophilic polymers), preferably less than 80 g/m², more preferably less than 50 g/m² and even more preferred less than 35 g/m², when measured according to the Cylinder Centrifuge Retention Capacity (CCRC) Test Method described below in detail. Preferably, the retention capacity of at least on region having dimensions of 5 cm by 10 cm the nonwoven web of the invention is less than 100 g/mm², more preferably is less than 80 g/m², even more preferably is less than 50 g/m² and most preferably is less than 35 g/m². Moreover, in the most preferred embodiment, very region of the nonwoven web has the above defined retention capacities.

Contrary thereto, nonwoven webs comprising superabsorbent polymers of the prior art typically have retention capacities from about 800 g/m² to several thousands of g/m².

For comparison, untreated nonwoven webs having a basis weight from 5 g/m² to 200 g/m² and not comprising any hydrophilic polymers of the present invention commonly have retention capacities from about 1 g/m² to about 10 g/m²; nonwoven webs having a basis weight from 7 g/m² to 150 g/m² not comprising any hydrophilic polymers commonly have retention capacities from about 1 g/m² to about 7 g/m²; and nonwoven webs having a basis weight from 7 g/m² to 150 g/m² not comprising any hydrophilic polymers commonly have retention capacities from about 1 g/m² to about 5 g/m².

The retention capacity of the nonwoven web of the invention does not only depend on the add-on level of the hydrophilic polymers but also on the degree of cross-linking of these hydrophilic polymers: Polymers with a high degree of cross-linking commonly exhibit a lower absorption capacity and retention capacity. They are less swellable due to the relatively low molecular weight of the parts of the polymer between adjacent network cross-links. Therefore, highly cross-linked polymers are not able to swell as much as polymers which are cross-linked to a lower degree.

If the nonwoven web of the present invention is used in absorbent articles, it is desirable, that the nonwoven web exhibits low strike through times, even after subsequent gushes of liquid, such as urine. Liquid strike through refers to liquid passing through the nonwoven fabric with liquid strike through time referring to the time it takes for a certain amount of liquid to pass through the nonwoven web. Liquid strike through time according to the present invention is determined according to the test method set out below.

Preferably, the nonwoven web of the present invention exhibits a liquid strike through time of less than 5 seconds for the fifth gush of liquid with every gush comprising 5 ml of saline solution. More preferably, liquid strike through time is less than 4.5 seconds for a fifth gush, and even more preferably is less than 4.0 seconds for a fifth gush. Hence, liquid strike through is maintained even after several gushes. Further preferred, the liquid strike through time of said nonwoven web after the fifth gush does not increase by more than 5% compared to the strike through time of the first gush.

As the nonwoven web of the invention comprising hydrophilic polymers remains hydrophilic even after several weeks of storage (e.g., in a warehouse). Therefore, it is preferred, that the nonwoven web of the present invention exhibits a liquid strike through time of less than 5 seconds for the fifth gush of liquid even when the nonwoven web has been stored for at least 10 weeks before liquid strike through time is tested.

No meaningful part of the hydrophilic polymers is washed off when the nonwoven fabric is exposed to aqueous solvents. Therefore, no significant surface tension reduction occurs when nonwoven fabric is exposed to aqueous solutions.

Generally, if the nonwoven web of the invention is applied in absorbent articles, it is preferred that the surface tension of aqueous wash-off from the treated nonwoven fabric is at least 65 mN/m, more preferably at least 68 mN/m and even more preferably at least 71 mN/m.

Moreover, it is preferred, to have nonwoven webs, wherein the distribution of the hydrophilic polymers comprised by the nonwoven web of the invention is substantially homogenous. “Substantially homogeneous”, according to the present invention, is defined as follows: The hydrophilic polymers comprised by the nonwoven web, cover at least 60% of a randomly selected area of 100 mm² (10 mm by 10 mm) within the nonwoven web. The selected area is analyzed with scanning electron microscopy. More preferably, the hydrophilic polymers cover at least 70%, even more preferably by at least 80% of the randomly selected area of 100 mm². The selected area is analyzed using electron microscopy.

Preferred hydrophilic polymers of the present invention are partially neutralized poly(meth)acrylic acids, their copolymers and starch derivatives thereof. Most preferably, the hydrophilic polymers comprise polyacrylic acid (i.e., poly(sodium acrylate/acrylic acid)). Preferably, the hydrophilic polymers are neutralized to at least 50%, more preferably at least 70%, even more preferably from between 70% and 99%.

Generally, the degree of neutralization has certain impacts both on the nonwoven web and on the method according to the present invention: The non-neutralized, acid form of the monomer is easier to polymerize and the resultant cross-linked polymer networks swell less in water and physiological solutions. Moreover, polymers comprising mainly non-neutralized monomers are less hydrophilic and therefore, less desirable for the present invention.

Solutions comprising neutralized monomers (salt) do not evaporate easily and are more hydrophilic. Due to the higher hydrophilicity, they are also more difficult to apply on the—commonly hydrophobic—nonwoven webs/plurality of fibers.

The method of the present invention enables a very efficient polymerization process with a low amount of un-reacted monomers remaining on the web/plurality of fibers after polymerization. Preferably, the amount of un-reacted monomers after polymerization is less than 2000 ppm, more preferably less than 1000 ppm relative to the dry weight of the web/plurality of fibers. This is, e.g., due to the fact that homo-polymerization of the monomers is not detrimental for the invention as long as the components comprised by the aqueous solution, such as the monomers, initiator molecules and cross-linking molecules are in close contact with the fibers of the nonwoven web/plurality of fibers, thus enabling formation of cross-linked polymer “around” the fibers. This is contrary to processes, which focus on the polymers being chemically grafted onto the fibers, especially processes, which do not apply cross-linking molecules. In these processes, homo-polymerization of the monomers has to be avoided and graft polymerization onto the fibers has to be ensured (e.g., by selecting an appropriate initiator). As a consequence, the polymerization process is rather inefficient, resulting in a relatively high amount of residual, un-reacted monomers remaining on the nonwoven web/plurality of fibers after the UV radiation. Therefore, such processes commonly require a washing step after UV radiation to wash off the un-reacted monomers.

According to the present invention, the amount of un-reacted monomers remaining on the web/plurality of fibers after polymerization is preferably less than 2000 ppm, more preferably less than 1000 ppm. The amount of residual monomers is determined according to the Edana Test Method 410.2-02 of 2002 “Determination of the amount of residual monomers”, whereby the test method is modified such that instead of 1.000 g of PA superabsorbent powder, 1.000 g of nonwoven web is used as test portion.

Hence, a further advantage of the method of the invention is that the amount of un-reacted, residual monomers remaining on the nonwoven web/plurality of fibers after UV radiation is relatively low. Accordingly, the method of the present invention does not require a washing step after UV radiation, which is desirable for high speed nonwoven production processes. Un-reacted monomers are especially problematic, if the nonwoven web/plurality of fibers of the invention is applied in absorbent articles, because a high level of un-reacted monomers is considered undesirable, especially when they come into contact with the skin of the wearer.

### Absorbent Articles

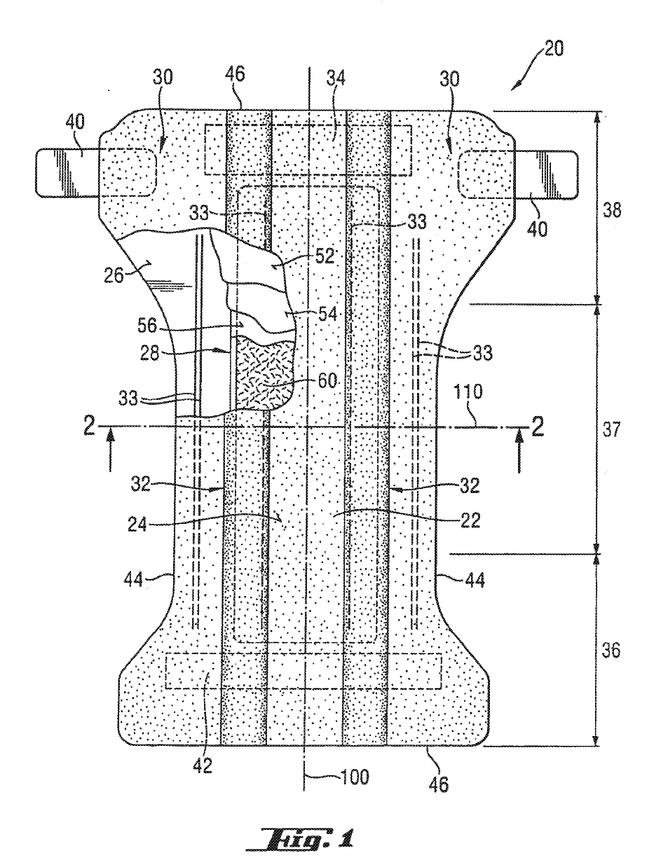

FIG. 1 is a plan view of a diaper 20 as a preferred embodiment of an absorbent article according to the present invention. The diaper is shown in its flat out, uncontracted state (i.e., without elastic induced contraction). Portions of the structure are cut away to more clearly show the underlying structure of the diaper 20. The portion of the diaper 20 that contacts a wearer is facing the viewer. The chassis 22 of the diaper 20 in FIG. 1 comprises the main body of the diaper 20. The chassis 22 comprises an outer covering including a liquid pervious topsheet 24 and/or a liquid impervious backsheet 26. The chassis 22 may also include most or all of the absorbent core 28 encased between the topsheet 24 and the backsheet 26. The chassis 22 preferably further includes side panels 30, leg cuffs 32 with elastic members 33 and a waist feature 34. The leg cuffs 32 and the waist feature 34 typically comprise elastic members. One end portion of the diaper is configured as the front waist region 36 of the diaper 20. The opposite end portion is configured as the rear waist region 38 of the diaper 20. The intermediate portion of the diaper is configured as the crotch region 37, which extends longitudinally between the front and rear waist regions. The crotch region 37 is that portion of the diaper 20 which, when the diaper is worn, is generally positioned between the wearer's legs.

The waist regions 36 and 38 may include a fastening system comprising fastening members 40 preferably attached to the rear waist region 38 and a landing zone 42 attached to the front waist region 36.

The diaper 20 has a longitudinal axis 100 and a transverse axis 110. The periphery of the diaper 20 is defined by the outer edges of the diaper 20 in which the longitudinal edges 44 run generally parallel to the longitudinal axis 100 of the diaper 20 and the end edges 46 run generally parallel to the transverse axis 110 of the diaper 20.

The diaper may also include other features as are known in the art including front and rear ear panels, waist cap features, elastics and the like to provide better fit, containment and aesthetic characteristics.

The absorbent core 28 may comprise any absorbent material that is generally compressible, conformable, non-irritating to the wearer's skin, and capable of absorbing and retaining liquids such as urine and other certain body exudates. The absorbent core 28 may comprise a wide variety of liquid-absorbent materials commonly used in disposable diapers and other absorbent articles such as comminuted wood pulp, which is generally referred to as air felt. Examples of other suitable absorbent materials include creped cellulose wadding; melt blown polymers, including co-form; chemically stiffened, modified or cross-linked cellulosic fibers; tissue, including tissue wraps and tissue laminates, absorbent foams, absorbent sponges, absorbent gelling materials, or any other known absorbent material or combinations of materials. The absorbent core may further comprise minor amounts (typically less than 10%) of non-liquid absorbent materials, such as adhesives, waxes, oils and the like.

Furthermore, the SAP particles of the present invention can be applied as absorbent materials. The SAP particles of the present invention preferably are present in amounts of at least 50% by weight of the whole absorbent core, more preferably at lest 60%, even more preferably at least 75% and still even more preferably at least 90% by weight of the whole absorbent core.

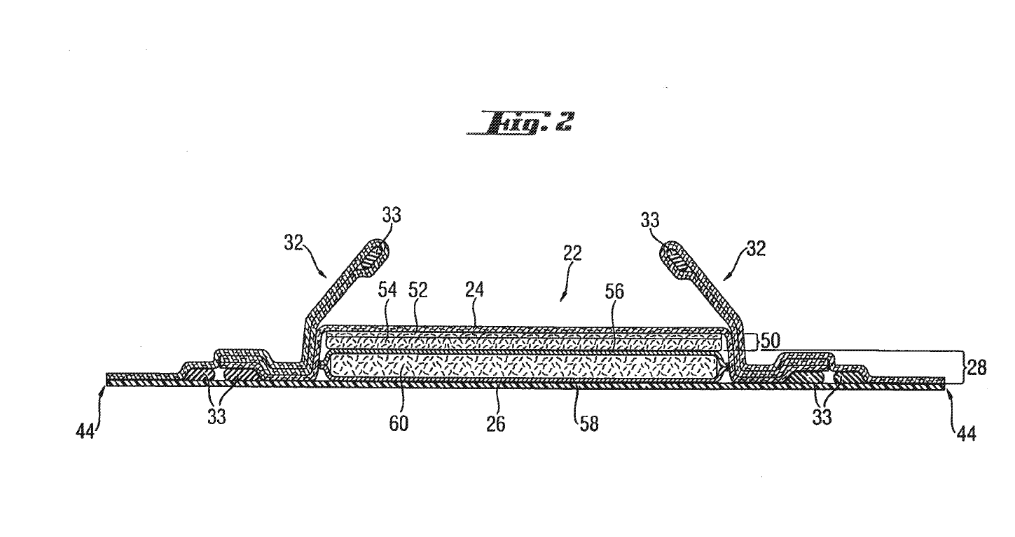

FIG. 2 shows a cross-sectional view of FIG. 1 taken in the transverse axis 110. In FIG. 2 illustrates a preferred embodiment of the different zones comprised by the absorbent cores. In FIG. 2, the fluid acquisition zone 50 comprises an upper acquisition layer 52 and a lower acquisition layer 54, while the fluid storage zone underneath the fluid acquisition zone comprises a storage layer 60, which is wrapped by an upper core wrap layer 56 and a lower core wrap layer 58.

In one preferred embodiment the upper acquisition layer 52 comprises a nonwoven fabric according to the present invention, whereas the lower acquisition layer 54 preferably comprises a mixture of chemically stiffened, twisted and curled fibers, high surface area fibers and thermoplastic binding fibers. In another preferred embodiment both acquisition layers 52, 54 are provided from a non-woven web according to the present invention. The acquisition layer preferably is in direct contact with the storage layer.

The storage layer 60 is preferably wrapped by a core wrap material. In one preferred embodiment the core wrap material comprises the nonwoven web of the present invention. The core wrap may comprise a top layer 56 (also called “core-cover”) and a bottom layer 58. The top layer 56 and the bottom layer 58 may be provided from two or more separate sheets of materials or they may be alternatively provided from a unitary sheet of material. If the top and bottom layers 56, 58 are provided from separate sheets, at least the top layer 56 preferably comprises the nonwoven web of the present invention, while the bottom layer 58 may comprise other nonwoven webs. In embodiments, where a unitary sheet of material is used, the unitary sheet may be wrapped around the storage layer 60, e.g., in a C-fold. Furthermore, a unitary sheet may be sealed after it has been wrapped around the storage layer.

The storage layer the present invention typically comprises SAP particles mixed with fibrous materials. Other materials as suitable for the absorbent core may also be comprised.

According to the present invention, preferably the topsheet 24 and/or all or a part of the core wrap of the absorbent article is made of the hydrophilic nonwoven fabric of the present invention. Moreover, the hydrophilic nonwoven fabric according to the present invention is preferably used as acquisition material 52 and/or 54 in the absorbent core 28.

### Test Methods

### 1. Cylinder Centrifuge Retention Capacity (CCRC)

This test serves to measure the saline-water-solution retention capacity of the nonwoven synthetic nonwoven fibrous web used herein, when the webs are submitted to centrifuge forces (and it is an indication of the maintenance of the absorption capacity of the nonwoven fibrous webs in use, when also various forces are applied to the material).

The test can be carried out with any nonwoven fibrous webs, e.g., with nonwoven fibrous webs not comprising any hydrophilic polymers or any coatings at all, with the nonwoven fibrous webs according to the present invention comprising hydrophilic polymers and with nonwoven fibrous webs comprising superabsorbent polymer particles as are known in the art.

First, a saline-water solution is prepared as follows: 18.00 g of sodium chloride is weighed and added into a two liter volumetric flask, which is then filled to volume with 2 liter deionised water under stirring until all sodium chloride is dissolved.

A pan with a minimum 5 cm depth, and large enough to hold four centrifuge cylinders is filled with part of the saline solution, such that up to a level of 40 mm (±3 mm).

Each nonwoven web sample is tested in a separate cylinder and each cylinder to be used is thus weighed before any sample is placed in it, with an accuracy of 0.01 g. The cylinders have a very fine mesh on the bottom, to allow fluid to leave the cylinder.

For each measurement, a duplicate test is done at the same time; so two samples are always prepared as follows:

0.3 g of a nonwoven web, which is to be tested, is weighed, with an accuracy of 0.005 g (this is the ‘sample’), and then the sample is transferred to an empty, weighed cylinder. (This is repeated for the replica.)

Directly after transferring the sample to a cylinder, the filled cylinder is placed into the pan with the saline solution (Cylinders should not be placed against each other or against the wall of the pan.).

After 15 min (±30 s), the cylinder is removed from the pan, and the saline solution is allowed to drain off the cylinder; then, the cylinder is re-placed in the pan for another 45 min. After the total of 60 minutes immersion time, the cylinder is taken from the solution and excess water is allowed to run off the cylinder and then, the cylinder with the sample is placed in the cylinder stands inside a centrifuge, such that the two replicate samples are in opposite positions.

The centrifuge used may be any centrifuge equipped to fit the cylinder and cylinder stand into a centrifuge cup that catches the emerging liquid from the cylinder and capable of delivering a centrifugal acceleration of 250 g (±5 g) applied to a mass placed on the bottom of the cylinder stand (e.g., 1300 rpm for a internal diameter of 264 mm). A suitable centrifuge is Heraeus Megafuge 1.0 VWR # 5211560 (VWR Scientific, Philadelphia, USA). The centrifuge is set to obtain a 250 g centrifugal acceleration. For a Heraeus Megafuge 1.0, with a rotor diameter of 264 mm, the setting of the centrifuge is 1300 rpm.

The samples are centrifuged for 3 minutes (±10 s).

The cylinders are removed from the centrifuge and weighed to the nearest 0.01 g.

For each sample (i), the cylinder centrifuge retention capacity (CCRC) Wᵢ, expressed as grams of saline-water-solution absorbed per gram of nonwoven web is calculated as follows:

$${w}_{i} = \frac{{m}_{CS} - ( {m}_{Cb} + {m}_{S} )}{{m}_{S}} [ \frac{g}{g} ]$$

wherein:

m_{CS}: is the mass of the cylinder with sample after centrifugation [g]

m_{Cb}: is the mass of the dry cylinder without sample [g]

m_{S}: is the mass of the sample without saline solution [g]

Then, the average of the two Wᵢ values for the sample and its replica is calculated (to the nearest 0.01 g/g).

Hence, for a nonwoven web having a basis weight of 15 g/m², the CCRC would be 15 times the CCRC value Wᵢ determined by the above formula. For example, for a nonwoven web having a basis weight of 15 g/m² and a CCRC of 5 g/g, the CCRC per square meter of web would be 15 g/m²×5 g/g=75 g/m².

### 2. Determination of Add-On Level

### a) Add-On Level of Hydrophilic Polymer on Nonwoven Web:

The add-on level is determined by weighing a sample of 1 m² of a nonwoven web, which does not comprise cross-linked hydrophilic polymers. The sample is weighed to an accuracy of ±0.01 g.

After the sample has been treated to comprise the hydrophilic polymers according to the invention, it is weighed again to an accuracy of ±0.01 g. The add-on level is then calculated as follows:

[(wₐ×100)/w_{b}]−100=% of add-on level with

wₐ: weight of web comprising hydrophilic polymers

w_{b}: weight of web without hydrophilic polymers

If the add-on level is determined after the web has been treated according to the method of the invention, it may be necessary to subject the web to a drying step before determining the add-on level, to ensure, that no residual aqueous solution is left on the nonwoven web test sample.

### b) Add-on Level of Hydrophilic Polymer on Plurality of Fibers:

The add-on level of monomers is determined analogue to the determination for the nonwoven web described above, but instead of weighing a sample of 1 m² of a nonwoven web, the initial test sample is 10 g of a plurality of fibers, wherein the 10 g of a plurality of fibers do not comprise cross-linked hydrophilic polymers. The sample is weighed to an accuracy of ±0.01 g.

After the sample has been treated to comprise the hydrophilic polymers according to the invention, it is weighed again to an accuracy of ±0.01 g. The add-on level is then calculated as follows:

[(wₐ×100)/w_{b})]−100=% of add-on level with

wₐ: weight of plurality of fibers comprising hydrophilic polymers

w_{b}: weight of plurality of fibers without hydrophilic polymers (=10 g)

If the add-on level is determined after the plurality of fibers has been treated according to the method of the invention, it may be necessary to subject the plurality of fibers to a drying step before determining the add-on level, to ensure, that no residual aqueous solution is left on the plurality of fibers test sample.

### 3. Determination of Surface Tension

The surface tension (unit: mN/m) is determined according to the following test.

### Apparatus:

Equipment: K10 tensiometer provided by Krüss GmbH, Germany or equivalent. The vessel elevation speed should be 4 min/min. Liquid surface height should be sensed automatically when using a plate or a ring. The equipment must be able to adjust the sample position automatically to the correct height. Precision of test should be +/−0.1 mN/m.

### Procedure:

1. Pouring 40 ml of saline (0.9 wt % NaCl in deionized water) into a cleaned beaker.
2. Testing the surface tension with a platinum ring or a platinum plate. The surface tension should be 71-72 mN/m at 20° C.
3. Cleaning the beaker with deionized water and isopropanol and burning it out with a gas burner for a few seconds. Waiting until equilibrate to room temperature is reached.
4. Placing 10 60×60 mm pieces of test nonwoven into a cleaned beaker. The nonwoven should have a basis weight of at least 10 g/m².
5. Adding 40 ml of saline (0.9 wt % NaCl in deionized water).
6. Stirring with a clean surfactant-free plastic stick for 10 seconds.
7. Letting the solution with nonwoven stand for 5 minutes.
8. Stirring again for 10 seconds.
9. Removing the nonwoven from the solvent with a clean surfactant-free plastic stick.
10. Letting the solution stand for 10 minutes.
11. Testing surface tension with a platinum plate or platinum ring.

### 4. Determination of Strike Through

The test is carried out based on Edana Method 150.3-96 (February 1996) Liquid Strike Through Time. As a modification compared to the Edana Method, the test described below does not only measure the first gush but several subsequent gushes.

### Apparatus

### Lister Strike Through Equipment:

Funnel fitted with magnetic valve: Rate of discharge of 25 ml in 3.5 (±0.25) seconds
Strike through plate: Constructed of 25 mm thick acrylic glass. The total weight of the plate must be 500 g. The electrodes should be of non-corrosive material. The electrodes are set in (4.0 mm×7.0 mm) cross section grooves, cut in the base of the plate and fixed with quick setting epoxy resin.
Base plate: A square of acrylic glass 125 mm×125 mm approximately.
Ring stand to support the funnel
Electronic Timer measuring to 0.01 seconds
Burette with 50 ml capacity
Core filter paper Ahlström Grade 989 or equivalent (average Strike Through time 1.7 s+−0.3 s, dimensions: 10×10 cm)

### Procedure

1. Carefully cutting the required number of samples, 12.5 cm×12.5 cm with touching the sample only at the edge of the sample.
2. Taking 10 plies of core filter paper.
3. Placing one sample on the set of 10 plies of filter paper on the base plate. The sample should be positioned on the filter paper in such a way that the side of the nonwoven, which is intended to face the user's skin (when applied in an absorbent article) is uppermost.
4. Placing the strike through plate on top with the center of the plate over the center of the test piece.
5. Centering the burette and the funnel over the plate.
6. Ensuring that the electrodes are connected to the timer. Switching on the timer and set the clock to zero.
7. Filling the burette with saline solution (0.9 wt % NaCl in deionized water).
8. Keeping the discharge valve of the funnel closed and run 5.0 ml of liquid (═One gush) from the burette into the funnel.
8. Opening the magnetic valve of the funnel to discharge 5.0 ml of liquid. The initial flow of liquid will complete the electrical circuit and start the timer. It will stop when the liquid has penetrated into the pad and fallen below the level of the electrodes in the strike through plate.
9. Recording the time indicated on the electronic timer.
10. Waiting for 60 seconds and going back to point 6 for the second, the third gush and any subsequent gush, with each gush comprising 5 ml of liquid.
11. Report: Time for the 1^{st}, 2^{nd} and any subsequent gush in seconds.

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for treating a synthetic nonwoven fibrous web to comprise cross-linked hydrophilic polymers, the method comprising the steps of
a) providing a synthetic nonwoven fibrous web;
b) providing an aqueous solution comprising hydrophilic monomers, cross-linker molecules and radical polymerization initiator molecules;
c) contacting the web with the aqueous solution, such that the web is penetrated by the aqueous solution; and
d) exposing the web/plurality of fibers to UV radiation;
wherein the concentration of the monomers in the aqueous solution is selected such that the add-on level of the hydrophilic polymers on the nonwoven web is less than 30% by weight of the web without said hydrophilic polymers.

2. The method of claim 1, wherein the web is contacted with the aqueous solution by spraying, kiss-roll coating, or slot coating.

3. The method of claim 1, wherein the hydrophilic monomers comprise at least one unsaturated double bond.

4. The method of claim 1, wherein the aqueous solution comprises between 10% and 70% hydrophilic monomers, by weight of aqueous solution.

5. The method of claim 1, wherein the radical polymerization initiator molecules include two or more different initiators having maximum UV absorption at different ranges within the UV spectrum.

6. The method of claim 1, wherein the aqueous solution comprises between 0.01% and 5% radical polymerization initiator molecules, by weight of aqueous solution.

7. The method of claim 1, wherein the hydrophilic monomers are polyfunctional.

8. The method of claim 1, wherein the aqueous solution comprises between 0.01% and 10% cross-linker molecules, by weight of aqueous solution.

9. The method of claim 1, wherein the aqueous solution further comprises a surfactant.

10. The method of claim 1, wherein the web is contacted with the aqueous solution under inert gas atmosphere.

11. The method of claim 1, further comprising subjecting the web to corona treatment.

12. The method of claim 1, wherein the web is exposed to UV radiation on each of two major surfaces.

13. The method of claim 1, wherein the step of exposing the web to UV radiation is carried out under inert gas atmosphere.

14. The method of claim 1, further comprising drying the web.

15. The method of claim 1, wherein the hydrophilic polymers are substantially homogenously distributed on the web.

16. The method of claim 1, wherein the hydrophilic polymers are selected from the group consisting of partially neutralized poly(meth)acrylic acids, copolymers of partially neutralized poly(meth)acrylic acids, and starch derivatives of partially neutralized poly(meth)acrylic acids.

17. The method of claim 1, wherein the amount of un-reacted monomers remaining on the web after exposure to UV radiation is less than 1000 ppm, relative to the dry weight of the web.

18. An absorbent article comprising a web formed according to the method of claim 1.

19. The absorbent article of claim 18, wherein the web is used as one or more of the components selected from the group consisting of a topsheet, all or part of a core wrap, an acquisition material, an absorbent core, and combinations thereof.

20. The absorbent article of claim 19, wherein the web has a retention capacity less than 100 g/m².
